(19) United States
(12) Patent Application Publication
(10) Pub. No.: US 2008/0058664 A1
(43) Pub. Date: Mar. 6, 2008

(54) Patient event recording and reporting apparatus, system, and method

(75) Inventor: Emily Mirro, San Francisco, CA (US)

Correspondence Address: MORRISON & FOERSTER LLP, 755 PAGE MILL RD, PALO ALTO, CA, 94304-1018, US

(73) Assignee: NeuroPace, Inc., Mountain View, CA (US)

(21) Appl. No.: 11/512,799

(22) Filed: Aug. 29, 2006

(51) Int. Cl. A61B 5/04

(52) U.S. Cl. 600/523

(57) ABSTRACT

According to various embodiments, a portable, patient-triggered device to record symptoms experienced by a patient along with time and date, and a bidirectional data link to communicate with an implantable medical device to download corresponding electrographic signal records and/or diagnostic data for later relay to a server are presented. According to some embodiments, the portable device can also deliver visual and audio queries to the patient on a scheduled basis to encourage compliance with a treatment regimen. According to an embodiment, the data link between the portable device and the implantable medical device is wireless. According to various other embodiments, the data link between the portable device and the server can be wired or wireless. The portable device can be configured as a watch, a key fob, a pendant, or the like.

### CROSS-REFERENCE TO RELATED APPLICATIONS

This application is related to copending U.S. utility applications Ser. No. 10/060,045, filed Jan. 29, 2002, Ser. No. 10/095,892, filed Mar. 12, 2002, and Ser. No. 10/941,759, filed Sep. 14, 2004, each of which is hereby incorporated herein by reference in the respective entirety of each.

### TECHNICAL FIELD

Embodiments of the present invention relate to devices for recording symptomatic events by patients for reporting. In particular, some embodiments relate to such devices that can be communicatively linked with implantable medical devices (IMDs) to capture correlated IMD data for subsequent reporting.

### BACKGROUND

Epilepsy, a neurological disorder characterized by the occurrence of seizures (specifically episodic impairment or loss of consciousness, abnormal motor phenomena, psychic or sensory disturbances, or the perturbation of the autonomic nervous system), is debilitating to a great number of people. It is believed that as many as two to four million Americans may suffer from various forms of epilepsy. Research has found that its prevalence may be even greater worldwide, particularly in less economically developed nations, suggesting that the worldwide figure for epilepsy sufferers may be in excess of one hundred million.

Because epilepsy can be characterized by seizures, its sufferers are frequently limited in the kinds of activities they may participate in. Epilepsy can prevent people from driving, working, or otherwise participating in much of what society has to offer. Some epilepsy sufferers have serious seizures so frequently that they are effectively incapacitated.

Furthermore, epilepsy is often progressive and can be associated with degenerative disorders and conditions. Over time, epileptic seizures may become more frequent and more serious, and in particularly severe cases, are likely to lead to deterioration of other brain functions (including cognitive function) as well as physical impairments.

The current state of the art in treating neurological disorders, particularly epilepsy, typically involves drug therapy and surgery. The first approach is usually drug therapy.

A number of antiepileptic drugs are approved and available for treating epilepsy, such as sodium valproate, phenobarbital/primidone, ethosuximide, gabapentin, phenytoin, and carbamazepine, as well as a number of others. Unfortunately, these antiepileptic drugs typically have serious side effects, especially toxicity, and it is extremely important in most cases to maintain a precise therapeutic serum level to avoid breakthrough seizures (if the dosage is too low) or toxic effects (if the dosage is too high).

Moreover, while many patients respond well to drug therapy alone, a significant number (at least 20-30%) do not. For those patients, surgery is presently the best-established and most viable alternative course of treatment.

Currently practiced surgical approaches include radical surgical resection such as hemispherectomy, corticectomy, lobectomy and partial lobectomy, and less-radical lesionectomy, transection, and stereotactic ablation. Besides being less than fully successful, these surgical approaches generally have a high risk of complications, and can often result in damage to eloquent (i.e., functionally important) brain regions and the consequent long-term impairment of various cognitive and other neurological functions. Furthermore, for a variety of reasons, such surgical treatments are contraindicated in a substantial number of patients. Unfortunately, even after radical brain surgery, many epilepsy patients are still not seizure-free.

Electrical stimulation is an emerging therapy for treating epilepsy. However, many currently approved and available electrical stimulation devices apply continuous electrical stimulation to neural tissue surrounding or near implanted electrodes, and most do not perform detection-they are not responsive to relevant neurological conditions.

A typical epilepsy patient experiences episodic attacks or seizures, which are generally electrographically defined as periods of abnormal neurological activity. As is traditional in the art, such periods are often referred to as “ictal”.

It is generally preferable to be able to detect and treat a seizure at or near the initial onset, or even before the onset. However, it is important to note that there are two general varieties of seizure onsets. A “clinical onset” represents the beginning of a seizure as manifested through observable clinical symptoms, such as involuntary muscle movements or neurophysiological effects such as lack of responsiveness. An “electrographic onset” refers to the beginning of detectable electrographic activity indicative of a seizure.

Movement disorders, i.e., neurological diseases or other problems that result in movement or muscle control problems are debilitating to a great number of individuals worldwide. In general, various movement disorders can be characterized by involuntary movement, impaired ability to move, or improper muscle tone.

Parkinson's Disease is generally characterized by tremor, an involuntary movement of the limbs and extremities that leads to an inability to perform normal daily life activities. It is believed that the symptoms of Parkinson's Disease are caused at least in part by a loss of dopaminergic neurons in the substantia nigra, a brain structure with an inhibitory effect on movement. Other symptoms of Parkinson's Disease include rigidity (undesired increased muscle tone, often leading to a “locking” effect in the limbs) and bradykinesia (slower-than-desired movements, and difficulty in initiating movements).

Essential Tremor, as its name suggests, can also be characterized primarily by tremor in the limbs and extremities. Tremor can also result as a symptom of Multiple Sclerosis and other diseases and disorders.

Other movement disorders are characterized by different symptoms. Dyskinesias, such as Huntington's Chorea, result in other forms of unwanted movement. Huntington's Chorea, in particular, is a congenital disorder that causes undesired “dance-like” movements of the limbs. It is believed to be caused by degeneration of the striatum. Hemiballismus, another dyskinesia, causes flailing of the limbs on one side of the body and is believed to be caused by degeneration of the subthalamic nucleus.

While drug therapies can provide good results for a substantial number of patients suffering from various movement disorders, particularly in the early stages before the disorders have progressed, there are some disadvantages to using drugs. In particular, patient compliance is particularly difficult to achieve when complex drug regimens are necessary to maintain an effective serum concentration. If drug levels are too low, the therapy may be ineffective; high levels can be damaging—they may cause serious side effects or even exacerbate the patient's movement disorders.

Surgery has also shown some promise and is effective with some patients, especially since there are fewer ongoing patient compliance issues (although patients who have had resective brain surgery are frequently kept on drug therapy as well). For example, lesions can be produced in the thalamus, globus pallidus, and other brain structures in an attempt to regulate patients'symptoms. However, clearly, resective brain surgery is irreversible and risky neurological deficits have been known to occur.

Continuous deep brain stimulation, particularly in the ventralis intermedius (VIM) nucleus of the thalamus, also has been shown to provide some relief from the symptoms of various movement disorders. However, this approach has resulted in some unpleasant side effects, in particular paresthesias, numbness, and slurring of speech. Moreover, a relatively small implantable device capable of performing continuous stimulation would tend to have a shorter battery life than would be desirable. Unlike other surgical treatments, continuous deep brain stimulation is reversible, in the event the side effects or neurological deficits resulting therefrom are more debilitating or unpleasant than the movement disorder.

Deep brain recordings from patients with tremor have shown an abnormal rhythmic electrical activity in the thalamus, globus pallidus, and subthalamic nucleus at a frequency of approximately 3-5 Hz. This rhythmic activity is associated with tremor, i.e., there is a substantially constant frequency and phase relationship between tremor and the electrophysiological activity. When electrical stimulation is applied in this same region of the brain where the 3-5 Hz signal is detected, the involuntary motion can be eliminated or at least moderated. Applying an electrical signal at 30-180 Hz using 300 microsecond biphasic pulses has been shown to eliminate or attenuate tremor. Stimulation by deep brain electrodes at 60-70 Hz using 300 microsecond biphasic pulses at 3-6 volts has been shown to cause a reduction in spasticity thereby allowing more normal movements.

Severe affective and behavioral psychiatric disorders affect 5 to 10 million adults in the United States and are the leading cause of disability in North America and Europe. Men and women of all ages and races are at risk for mental illness and for the associated morbidity and societal cost. Although psychopharmacological therapy provides at least partial relief for between 70 to 90% of persons suffering from major depression, bipolar disorder (BPD), obsessive-compulsive disorder (OCD) and panic and other severe anxiety disorders; others are not helped or experience unacceptable medication related side effects. Those experiencing schizophrenia, episodic behavioral disorders, post-traumatic stress disorder (PTSD), addictions, and the behavioral and social disorders associated with autism and pervasive developmental disorders are less often helped by pharmacotherapy or psychotherapy. The economic cost of untreated mental illness is more than 100 billion dollars each year in the United States.

Accordingly, new treatments are clearly needed for those whose symptoms persist and for those not tolerating therapy, as well as to relieve the societal burden created by untreated and undertreated mental illness.

Major depression is a serious and persistent medical illness affecting about 9.9 million American adults, or approximately 5 percent of the adult population in a given year. Among all medical illnesses, major depression is the leading cause of disability in the U.S. and many other developed countries. About three-fourths of those who experience a first episode of depression will have at least one other episode in their lives and some individuals have several episodes in the course of a year. If untreated, episodes can last anywhere from six months to a year. Left untreated, depression can lead to suicide.

Treatment options can include includes medications, psychotherapy, and electroconvulsive therapy (ECT) used alone or in combination. Although mild to moderate depression can often be treated successfully with medications or psychotherapy alone, severe depression usually requires a combination of psychotherapy and medication. ECT is highly effective for treatment resistant or treatment intolerant severe depression and for relieving symptoms such as psychosis or thoughts of suicide. However, ECT often requires repeated therapies and can cause persistent and troubling memory disturbances.

Bipolar disorder is another other common major psychiatric disorders that may be treatment resistant. Bipolar disorder is a chronic disorder that affects around 2.3 million adult Americans. Bipolar disorder can be characterized by episodes of mania and depression that can last from days to months. Persons with bipolar disorder usually require lifelong treatment, and recovery between episodes is often poor. Generally, those who suffer from bipolar disorder have symptoms of both mania and depression (sometimes at the same time). Medications are available to treat depression or mania and provide mood stabilization. However, most persons with bipolar disorder require multiple medications to achieve symptom relief. Thus, persons with bipolar disease are at risk for medication related side effects that prompt some to discontinue therapy. Others who are compliant with therapy do not achieve complete symptom relief.

Obsessive-Compulsive Disorder (OCD) affects approximately 2 to 3% of the population as confirmed in the U.S. and international epidemiological studies, and is two to three times more common than schizophrenia and bipolar disorder. Obsessions and compulsive behaviors can cause suffering and severe restrictions on life activities. Response to treatment varies from person to person. Most people treated with effective medications find their symptoms reduced by about 40 percent to 50 percent. Although such symptom relief is welcome, freedom from symptoms is rarely achieved and only a small number of people are fortunate to go into total remission. Only one fifth of patients achieve full remission within one decade of the onset of the illness and two-thirds continue to experience symptoms despite treatment with selective serotonin reuptake inhibitor drugs (SSRIs) and the use of behavior therapy.

Some persons with chronic, treatment resistant mental illness have turned to surgical therapies. Frontal lobotomy was practiced in the last century. Although effective in some cases, the surgery was crude, not standardized and involved destruction of a large region of the frontal lobe. The procedure was largely abandoned because of unacceptable surgical complications and because of ethical violations in its application. A few centers continued to offer surgical therapy to the most devastated patients. The National Commission for the Protection of Human Subjects of Biomedical and Behavioral Research (1977) indicated that more than half of 400 surgeries performed annual between 1971 and 1973 for psychiatric indications were efficacious, and there is reason to believe efficacy has improved since then.

Recently, neurosurgeons have developed more precise surgical procedures to treat psychiatric disorders, including depression and, more commonly, obsessive-compulsive disorder. The majority of these procedures involve targeted ablative procedures. In these refractory patients, stereotactic surgical interventions performed include subcaudate tractotomy, limbic leucotomy, capsulotomy, and cingulotomy. Cingulotomy is the most commonly performed procedure. Twenty-five to 30% of patients treated with cingulotomy experience improvement at more than 2 years follow-up. However, these procedures are associated with risks including changes in personality and development of epilepsy. Other adverse effects include frontal lobe deficit in as many as 30% with fatigue, emotional blunting, emotional incontinence, indifference, low intiaitive, disinhibition and impaired judgment. These procedures carry the risk that the lesion will be malpositioned, which may require repeated surgery to extend the size of the lesion. Thus, concerns about safety and the irreversibility of surgical procedures remain.

Due to the limited response to lesion based surgery and concerns about adverse effects, some investigators have turned to electrical stimulation therapy. Building on the experience from essential tremor and Parkinson's Disease, investigators have utilized commercially available deep brain stimulators implanted in the anterior internal capsule bilaterally and have reported symptomatic improvement in OCD. However, because of the stimulation requirements for clinical response (4 to 10.5V, impedance 700 ohms, pulse width 210 microseconds, 100 Hz frequency) the stimulator battery requires replacement every 5 to 12 months, limiting patient acceptance for this therapy.

The neuroanatomical base for many psychiatric disorders is better understood because of advances in functional neuroimaging, such as Positron Emission Tomography (PET), Magnetic Resonance Imaging (MRI), Functional MRI (fMRI), and Magnetoencephalography (MEG). In addition, clinical observations after destructive brain lesions identify regions subserving specific aspects of behavior and affect. The cingulate cortex is a large structure around the rostrum of the corpus callosum that has extensive projections with the amygdala, periaqueductal grey, ventral striatum, orbitofrontal and anterior insular cortices. This structure and its interconnections are intimately involved in mood and behavior. Dysfunction of the cingulate and disruption of its connections has been implicated in a number of psychiatric disorders. As noted above, cingulotomy is the most common psychosurgery procedure for major depression and obsessive-compulsive disorder. This procedure is effective for many but carries considerable risk for post-surgical changes in personality and motivation, and for post-operative epilepsy.

The size and complexity of the cingulate cortex poses a challenge in targeting the region responsible for specific psychiatric and behavioral disorders. The cingulate is divided functionally into regions concerned with affect and cognition. Affect is mediated in cingulate regions 25, 33 and rostral area 24 that are extensively interconnected to the amygdala and periaqueductal grey, as well as autonomic brainstem nuclei. The cognitive division resides in caudal areas 24′ and 32′, and in cingulate motor areas in the cingulate sulcus and nociceptive cortex. Individuals with disturbances to the cingulate cortex, such as those with cingulate onset epilepsy, often display sociopathic behavior. Elevated anterior cingulate activity may contribute to tics, obsessive-compulsive behaviors and aberrant social behavior. Reduced cingulate activity can contribute to schizophrenia, behavioral disorders such as akinetic mutism, diminished self-awareness and depression, motor neglect and impaired initiation of movement, reduced pain response and abnormal social behavior.

Accordingly a responsive implantable system capable of ameliorating the symptoms of, and in some cases the underlying causes of, various epileptic, movement, and psychiatric disorders is desirable. Furthermore, correlation of measurements and responses by such systems with symptomatic data reported by patients, for reporting purposes is also desirable.

U.S. Pat. No. 5,752,976 issued to Duffin, et al. on May 19, 1998 describes a wireless telemetry system for an implantable medical device (IMD), but teaches no provision for a patient's reporting of symptoms experienced. United States published application 2003/0144711A1 teaches systems and methods for communicating wirelessly with an implantable medical device, and relaying data to and from a central server, but does not teach a way for a patient to initiate implantable medical device recordings triggered by symptom reporting. Other current methods of patient symptom event reporting (such as seizures, pain, headaches, etc.) include patient event diaries (paper) to record the time/date, type and severity of events, magnets that can be swiped over the implantable medical device to mark clinical events experienced by the patient and patient data transmitters that can communicate with the implanted device to gather device recorded data.

### BRIEF SUMMARY OF EMBODIMENTS OF THE INVENTION

Described here are various embodiments including a portable, patient-triggered device to record events experienced by the patient along with time and date of the events, and a bidirectional data link to communicate with an implantable medical device to download the electrographic and diagnostic data corresponding to the patient-reported events for later relay to a server. According to some embodiments, the portable device can also deliver visual and audio queries to the patient on a scheduled basis to encourage compliance with a treatment regimen and to gather daily survey data of the patient's health. According to an embodiment, the data link between the portable patient-triggered device and the implantable medical device is wireless. According to various other embodiments, the data link between the portable patient-triggered device and the server can be wired or wireless. The portable patient-triggered device can be configured as a watch, a key fob, a pendant, or the like.

According to an embodiment of the invention, a patient event recording device (PER) comprises: a first switch; control logic operably coupled with the first switch; a transmitter operably coupled with the control logic, the transmitter configurable to transmit to an implantable medical device responsive to an actuation of the first switch; a receiver operably coupled with the control logic, the receiver configurable to receive transmissions from the implantable medical device, the received transmissions encoding medical device data; a memory operably coupled with the control logic, the memory configurable to record at least a portion of the medical device data; a first data communication interface operably coupled with the control logic, the first data communication interface configurable for coupling with a second data communication interface to relay the recorded data to a server; and a power source operably configured to power the patient event recording device.

According to a further embodiment, the medical device data comprises an electrographic record as recorded by the implantable medical device and diagnostic data logged by the implantable medical device.

The device may further comprise a charger interface operably coupled to the power source, and the power source comprises a rechargeable power cell or a clock typically operably coupled with the control logic. The clock is configurable to provide a time indication corresponding to the actuation of the first switch, and a memory is further configurable to record the time indication. A modem interface may be used to relay the time indication to the server.

The device may further comprise: at least one additional switch operably coupled to the control logic; and a video display operably coupled to the control logic. According to some embodiments the video display is configurable to display a time indication. According to other embodiments the video display is configurable to display a prompt corresponding to a scheduled time indication, wherein the displayed prompt comprises at least one of (i) a symptom question, (ii) a medication question, and (iii) an activity question.

According to some embodiments, the control logic is further configurable to store the time indication and an indication of an actuation of at least one of the first switch or the at least one additional switch subsequent to the display of the prompt. According to other embodiments the video display is configurable to display a prompt responsive to an actuation of at least one of the first switch or the at least one additional switch, wherein the displayed prompt comprises at least one of (i) a symptom question, (ii) a medication question, and (iii) a survey question. According to further embodiments, the control logic is further configurable to store an indication of an actuation of at least one of the first switch or the at least one additional switch subsequent to the display of the prompt.

The video display can be also configurable to display a prompt indicating an alarm condition consisting of at least one of (i) a scheduled server communication time indication, (ii) a scheduled medication administration time indication, (iii) a scheduled survey administration time indication, (iv) a memory capacity limit indication, and (v) a low power source indication.

The device may further comprise an audio generator operably coupled to the control logic, wherein the control logic is further configurable to actuate the audio generator indicating an alarm condition consisting of at least one of (i) a scheduled server communication time indication, (ii) a scheduled medication administration time indication, (iii) a scheduled survey administration time indication, (iv) a memory storage capacity limit indication, and (v) a low power source indication.

In general, methods of operating a patient event recording device (PER) are described, the methods comprise: recording a time indication, corresponding to a first switch actuation, in a memory; transmitting a first signal to an implantable medical device (IMD), the transmission being responsive to the first switch actuation; receiving a second signal from the implantable medical device, the reception being responsive to the transmission of the first signal, the second signal encoding data comprising at least one of (i) a representation of an electrographic record recorded by the IMD, and (ii) diagnostic data for the IMD; and storing at least a portion of the encoded data in the memory. According to another embodiment, the PER can transmit a signal to the IMD indicating that at least a portion of data stored on the IMD should be protected (i.e. not deleted or written-over).

The method may further comprise a visual display prompting a response to the indication of the first switch actuation wherein the displayed prompt comprises at least one of (i) a symptom question, (ii) a medication question, and (iii) a survey question; storing an indication of at least one switch actuation subsequent to the display of the prompt.

According to some other embodiments, the method further comprises: transmitting the time indication and at least a portion of the at least a portion of the encoded data that are stored in the memory to a server; and/or receiving management data from the server and storing at least a portion of the management data in the memory; and/or displaying an indication of the stored management data on a video display. Some embodiments further comprise actuating an audio generator responsive to the stored management data. Some embodiments further comprise transmitting a signal to the implantable medical device indicating that a least a portion of data stored on the implantable medical device should be protected.

Also described in general are systems for recording and reporting patient events, comprising a patient event recorder comprising control logic, a memory, a clock, a transceiver configurable for bidirectional data communication with an implantable medical device, a modem interface, a power source, and at least one switch; the implantable medical device, comprising a transceiver configurable for bidirectional data communication with the patient event recorder; and a docking adapter, comprising a modem or data communication circuit configurable for bidirectional data communication with the data communication interface of the patient event recorder.

According to some system embodiments, the power source of the patient event recorder comprises a rechargeable power cell, the patient event recorder further comprises a charger interface operably coupled to the rechargeable power cell, and the docking adapter further comprises a charger configurable to charge the power cell via the charger interface.

According to some system embodiments, the modem is one of (i) a phone line modem, (ii) an Ethernet® adapter, (iii) a universal serial bus modem, (iv) a Bluetooth® modem; (v) an IEEE802® Standards-series modem, a (vi) TIA® Standards-based wireless data modem, and (vii) a 3GPP® Standards-based wireless data modem.

According to some system embodiments, the transceivers of both the implantable medical device and the patient event recorder are in compliance with one of (i) Bluetooth® Standards, and (ii) FCC regulations for 433 MHz ISM band operation. Additional embodiments comprise a server configured for bidirectional communication with the modem.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

FIG. 1 illustrates a system environment according to an embodiment of the invention.

FIG. 2 is a block diagram according to an embodiment of the invention.

FIG. 3 illustrates an embodiment of the invention in a wrist watch configuration.

FIG. 4 illustrates an embodiment of the invention in a key fob configuration.

FIG. 5 illustrates a button arrangement according to an embodiment of the invention.

FIGS. 6 through 8 illustrate video displays according to various embodiments of the invention.

FIG. 9A illustrates another embodiment of the invention in a wrist watch configuration.

FIG. 9B illustrates a docking station according to an embodiment of the invention.

FIG. 10 is a control logic block diagram according to an embodiment of the invention.

The figures provided are merely representational and may not be drawn to scale. Certain proportions thereof may be exaggerated, while others may be minimized. The figures are intended to illustrate various embodiments of the invention that can be understood and appropriately carried out by those of ordinary skill in the art. Commonly designated elements among the various figures refer to common or equivalent elements in the depicted embodiments. The figures are not intended to be exhaustive or to limit the invention to the precise form disclosed. It should be understood that the invention can be practiced with modification and alteration, and that the invention be limited only by the claims and the equivalents thereof.

### DETAILED DESCRIPTION OF EMBODIMENTS OF THE INVENTION

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in applications, published applications and other publications that are herein incorporated by reference, the definition set forth in this section prevails over the definition that is incorporated herein by reference.

As used herein, a or an means at least one or one or more.

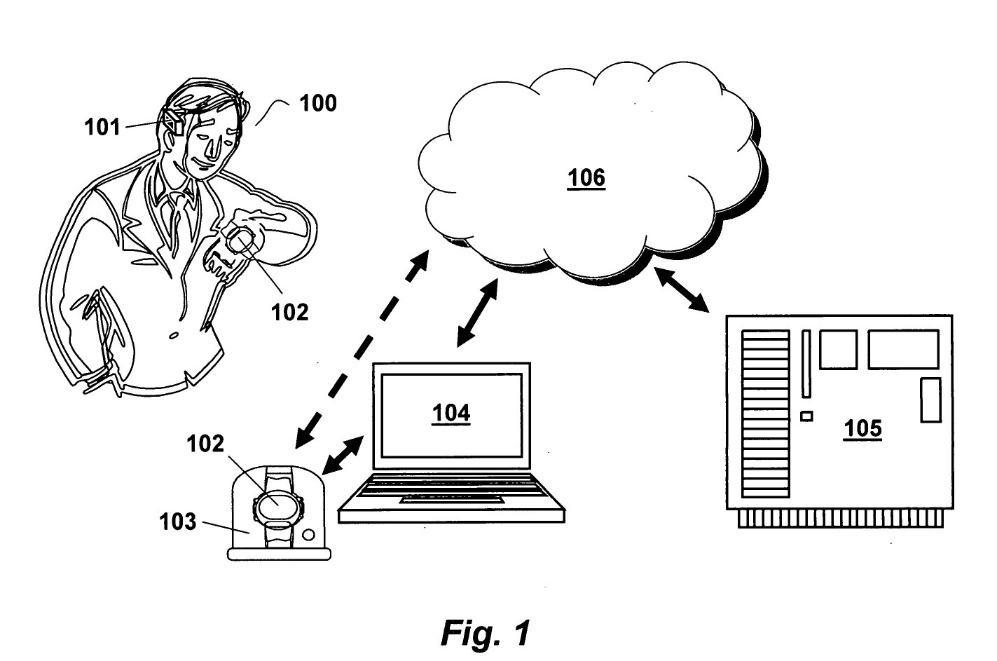

Embodiments of the Patient Event Recorder (PER) are intended to ease event data capture burden on both patients with implanted devices and their treating clinicians. FIG. 1 illustrates a system environment for a PER embodiment. Patient 100 has an implanted medical device (IMD) 101. IMD 101 is illustrated as a cranial implant, such as could be the case, for example, for a cranial neurostimulator IMD. According to other embodiments of the invention, the IMD could be another type of implantable medical device, for example, a cardiac device or the like. According to various embodiments of the invention, IMD 101 is battery powered and can also be equipped with sensors, stimulators, control logic, memory, an internal clock, and a transceiver for transdermal or transcranial wireless communication with an external device for provisioning and monitoring the IMD. Because surgery is usually required to replace the battery in an IMD, unless the battery is rechargeable, it is highly desirable to maximize battery life by minimizing battery discharge rate. Rechargeable batteries can be used in combination with a transdermally coupled charging device, however rechargeable batteries typically have lower gravimetric and volumetric stored energy densities, compared with non-rechargeable batteries—leading to a need for frequent charging and possibly size and weight penalties for the IMD.

Patient 101 is also shown wearing an embodiment of a PER 102, configured as a watch. Other configurations of PER 102 could be, for example, a key fob, a pendant, a box on a belt clip, or pocket or purse sized devices, to name a few. According to an embodiment of the invention, the PER can be equipped with a wireless communication transceiver (i.e., a transmitter and receiver combination) that is compatible with an IMD's transceiver, allowing for bidirectional communication between the PER and the IMD. The PER can give a patient the capability to record the time and date of their symptomatic events on an internal memory with the push of a button, as well as allow them to provide details on each of their events (type of event, severity of event, etc). The PER will also allow for collection of other, non-event related health information on a scheduled basis (daily medication logging, quality of life surveys, etc).

Various embodiments of the PER can communicate with an IMD with a variety of wireless technologies that are well known to one of ordinary skill in the art. For example, integrated circuits such as the ZARLINK™ ZL70100 (from ZARLINK™ Semiconductor, Inc.) operate in the 433 MHz ISM (Industrial, Scientific, and Medical band). Such receivers feature ultra low power consumption for long battery life and reliable body-wide communication range. Commercial transceiver ICs based on alternate wireless technologies such as BlueTooth® offer similar operating features.

When an event button is pressed on PER 102, the PER will send a command to IMD 101 to store the date/time of an event as well as an electrographic record of the event (if applicable). The PER will also upload (interrogate) data from the implanted device either upon command by the patient or when the event button is pushed; these capabilities will ensure that important device data is not overwritten. The PER can also signal the IMD to preserve stores of data within the IMD.

Referring again to FIG. 1, PER 102 is also shown installed in docking station 103. According to some embodiments, docking station 103 can directly communicate with a centralized server and database 105 via a network 106. According to some other embodiments, docking station 103 can communicate with centralized server and database 105 via an intermediate processor, illustrated as laptop 104. When installed in docking station 103, PER 102 can synchronize downloaded IMD data as well as the patient event data with the centralized database for access by the clinicians. The downloaded IMD data can comprise electrographic records captured by the IMD, as well as IMD self-diagnostic. The synchronization of the PER and centralized database will be performed by the patient using a PER docking station which is connected to the Internet. Clinicians will be able to send messages or commands to the PER (for patient review, among other purposes) using either a device programmer or through the centralized database.

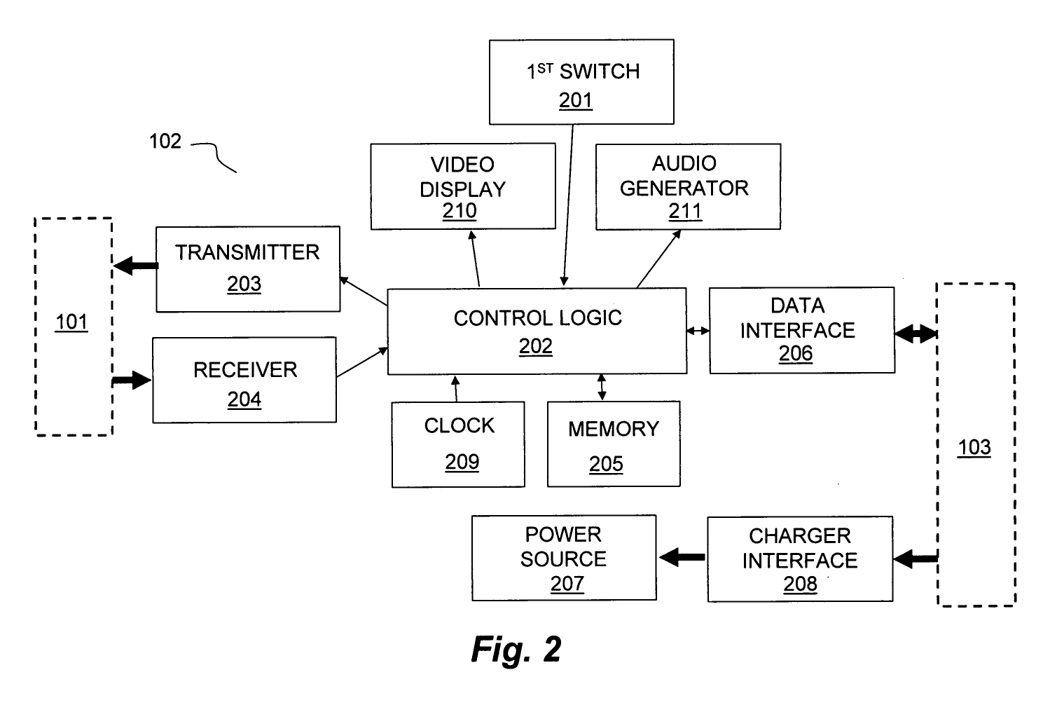

FIG. 2 illustrates a block diagram of a PER embodiment, along with couplings to IMD 101 and docking station 103. PER 102 is shown having a first switch 201 operably coupled with control logic 202 (described in more detail below). First switch 201 could be a momentary contact type pushbutton, or a resistive, capacitive, or optically sensitive type of finger proximity detector. According to some other embodiments, first switch 201 could be a motion or position sensor. According to still other embodiments, first switch 201 could be a speech or sound recognition device. In response to an actuation of first switch 201, control logic 202 can initiate several actions, including for example (i) causing transmitter 203 to transmit wake-up and/or other messages to IMD 101; (ii) causing receiver 204 to receive messages from IMD 101; (iii) causing a clock indication from clock 209 to be stored in memory 205; (iv) causing at least a portion of data received by receiver 204 to be stored in memory 205; and (v) causing video display 210 to display a prompt and/or causing audio generator 211 to announce a prompt. The type of prompts displayed will be discussed in further detail below in connection with PER applications. According to some embodiments an announced prompt can be a tone or series or combination of tones. According to some other embodiments, an announced prompt can be synthesized or recorded speech.

Power source 207 is operable configured to power the PER. According to some embodiments, power source 207 can be a primary battery such as a lithium oxide cell; a silver oxide cell, or the like. According to some other embodiments, power source 207 can be a fuel cell. According to still further embodiments, power source 207 can be a rechargeable battery or cell such as lithium-ion, lithium-polymer, nickel-metal hydride, nickel-cadmium, or the like. In embodiments where power source 207 is a rechargeable battery or cell, charger interface 208 is provided to interface with docking station 103 to provide for charging.

Data interface 206, operably coupled with the control logic, is configurable to couple with docking station 103 for the transfer of data to and from server/database 105 as described above in connection with FIG. 1. In order to provide for communication via network 106 (shown in FIG. 1), according to some embodiments, docking station 103 can comprise one of: (i) a phone line modem, (ii) an Ethernet® adapter, (iii) a universal serial bus, (iv) a Bluetooth® modem; (v) an IEEE802® Standards-series modem, a (vi) TIA® Standards-based wireless data modem, and (vii) a 3GPP® Standards-based wireless data modem

According to some embodiments additional switches can be coupled to control logic 202 to allow for patient responses to be entered into the PER. Examples of switch configurations will be discussed below. Generally speaking, configurations of fewer switches, each switch having a multiplexed function, may provide for more compact PER configurations, but configurations having more switches may provide for easier patient-user interfaces.

According to some embodiments, the video display is configurable to display a prompt corresponding to a scheduled time indication. Examples of such prompts include, without limitation, (i) a symptom question, (ii) a medication question, and (iii) a survey question. Typically a patient would then input a multiple choice response through one or more button actuations. Additionally, the actuation of a patient event switch could evoke the display of such prompts.

According to some embodiments the video display is configurable to display a prompt (and/or the audio generator is configurable to sound) indicating an alarm condition consisting of at least one of (i) a scheduled server communication time indication, (ii) a scheduled medication administration time indication, (iii) a scheduled survey question time indication, (iv) a memory capacity limit indication, and (v) a low power source indication.

According to a typical embodiment, the PER can have the following operating modes: (i) timekeeping (standard watch) mode; (ii) event recording mode; (iii) scheduled data collection mode; (iv) IMD interrogation mode; and (v) synchronization mode. Some functions, such as time and operational alarm functions would be operational regardless of operating mode.

The timekeeping mode may include standard watch functions and features such as time display, date display, audible alarm, vibrating alarm, display backlight, water resistance, and so forth.

The event recording mode can be enabled by a patient's actuation of an event button, causing the date and time of the event to be stored and communicated to the IMD. Thereafter, the video display would ask a question of the patient regarding the type and severity of the event. According to an exemplary embodiment, the patient would be provided with buttons designated one through ten and “A” through “I,” for example, to respond. According to further embodiments, the display may provide additional instructions to the patient on how to properly report the event. In this mode, the display screen can toggle between time/date and event recording information.

According to an exemplary embodiment, in the scheduled data collection mode, questions may be presented to a patient on a scheduled basis (as determined by the PER programmer). The patient will answer the questions presented and the PER will record the patient responses. Examples of types of data that might be collected by the PER can include quality of life survey questions, pain rating scale questions, medication logging questions, and patient activity questions, to name a few. The PER can sound an audible alarm and/or display a visual alarm when a patient response to these questions is necessary.

According to an embodiment of the IMD interrogation mode, the PER can request and receive IMD data in response to the actuation of an event button, or on command by the patient.

In a synchronization mode, the PER is typically coupled to a docking adapter for synchronization with a server/database, as described above. Using data obtained from the PER, the server/database obtains IMD device diagnostics and electrographic data with embedded markers indicating the patient event time/date and type of event (if applicable). The centralized database may supply on-demand reports of patient events, event classifications and other scheduled data collection that has taken place. Also in the synchronization mode, management data can be sent from the server/database to the PER. Such management data can include changes in treatment regimen, or updates to questions/choices presented to the patient on the PER.

An alarm may be used to alert the patient when it is time to upload data to the centralized database. An alarm may prompt the patient to provide a response to survey questions or event questions. An alarm may be provided at a scheduled time to remind a patient of medication dosages and times. An alarm may alert the patient when the implanted device battery is running low to indicate that the battery either needs to be recharged (if rechargeable device) or if the battery must be replaced. The clinician will configure the PER set-up during an office visit using the device programmer. Examples of set-up options include time/date, alarms, event type descriptions, etc.

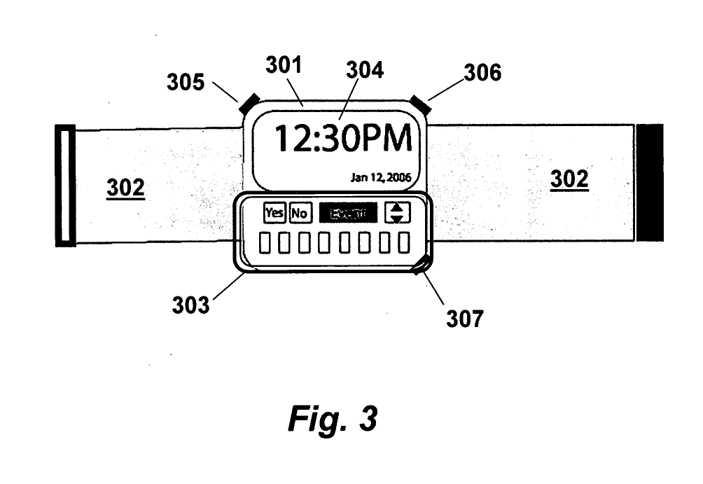

Turning now to FIG. 3, an embodiment of a PER in a wrist watch configuration is illustrated. Case body 301 can be formed of any appropriate polymer, metal, or metal alloy as is well known by one of ordinary skill in the watchmaking art. Wrist strap 302 is coupled to case body 301, any may be made of polymer, fabric, leather, or other material as is also well known by one of ordinary skill in the watchmaking art. Clear cover 303 protects an array of buttons on the face of case body 301, and may be attached to case body 301 by a hinge on the bottom, or on a side in various embodiments. Video display screen 304 is typically of the monochrome dot matrix liquid crystal display (LCD) type, possibly further comprising segmented and/or iconic display elements. In some embodiments, video display screen 304 can be illuminated by an electroluminescent panel, one or more light emitting diodes, incandescent lamps, or a cold cathode fluorescent tube to improve visibility in low ambient lighting conditions. In such embodiments, button 305 can be used to activate such light(s). According to other embodiments video display screen 304 could be of an active or passive color LCD display type. In this exemplary embodiment, button 306 can be used to initiate an interrogation of an IMD, and button 307 can be used to set the time and date. While buttons 305, 306, and 307 are shown on corners of the device, it should be understood that they could be placed in alternate locations, and that any desirable and suitable arrangement of buttons may be used.

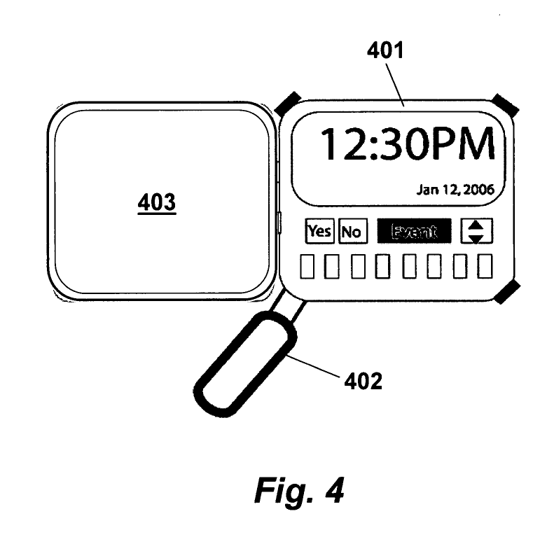

FIG. 4 illustrates an embodiment of a PER in key fob configuration. Construction and features are similar to the embodiment of FIG. 3, except for the inclusion of key ring 402, and now a clear protective cover 403 has been expanded to cover the entire face of the unit. In further embodiments a common PER in a case can be made reconfigurable as a watch or as a key fob by attaching a watch band or key chain, respectively. In yet another embodiment, the PER can be configured as a pendant by attaching a lanyard in place of the key ring.

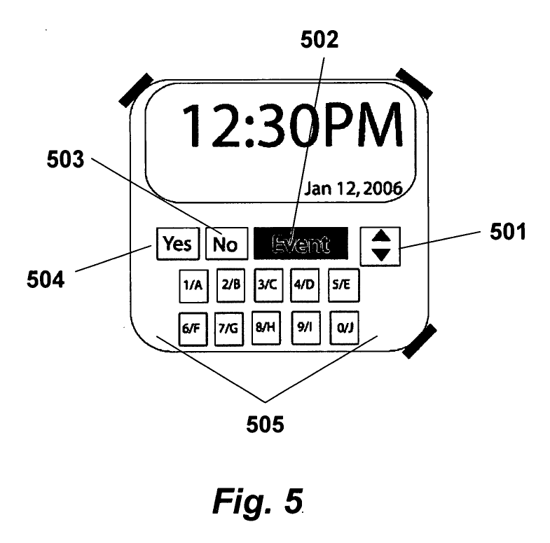

FIG. 5 illustrates a button arrangement detail according to an embodiment of the invention. Button 501 can be used to scroll the video display. Event marker button 502 is oversized for easier activation to signal a patient's symptom. Buttons 504 and 503 can be used to respond yes or no, respectively, to a question displayed on the video display. Ten button array 505 can be used to provide more detailed responses to questions. Again, any appropriate number, size, shape, color, alphanumeric markings, and arrangement of buttons may be used.

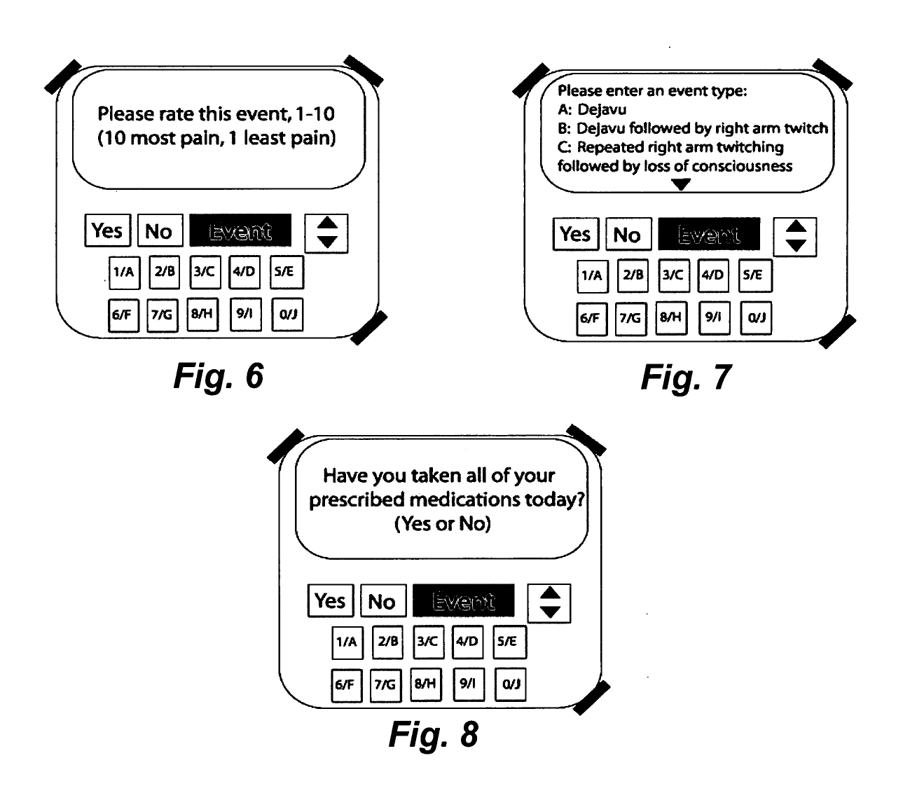

FIGS. 6 through 8 illustrate video displays according to various embodiments of the invention. FIG. 6 is an exemplary pain level reporting display. FIG. 7 illustrates a display requesting details about a seizure experience. FIG. 8 illustrates a display requesting verification of patient compliance with a medication regiment.

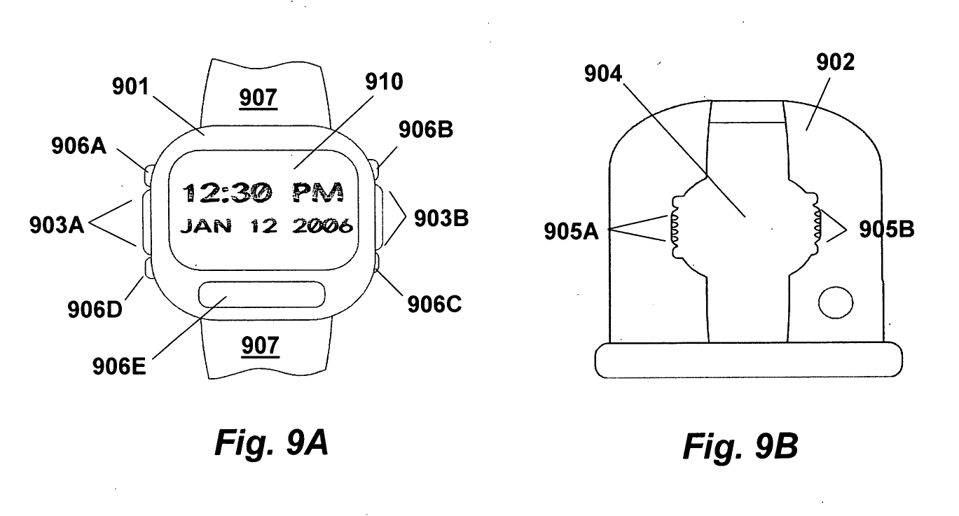

FIG. 9A illustrates another embodiment of the invention in a wrist watch configuration. According to this embodiment, PER case 901, band 907, and video display 910 are styled more like a traditional digital wrist watch. This embodiment uses only five push buttons, 906A, 906B, 906C, 906D, and 906E. In order to use fewer buttons, the button functions are more multiplexed, and the display is more menu-driven than in the above embodiments. Also shown are connector arrays 903A and 903B for coupling with a docking station. Again, any appropriate number, size, shape, color, alphanumeric markings, and arrangement of buttons may be used.

FIG. 9B illustrates a docking station according to an embodiment of the invention. Housing 902 has in indentation 904 into which the PER of FIG. 9A can be inserted. Connector arrays 905A and 905B are configured to couple with connector arrays 903A and 903B, respectively, of the PER of FIG. 9A.

Table 1 lists some examples of medical conditions for which various embodiments of the PER can be applied, along with respective uses.

TABLE 1

| Condition | Uses |
|---|---|
| Epilepsy | Record seizure event time/date |
| | Collect seizure event type and cluster information |
| | Store EEG for event |
| | Record medication logs |
| | Record monthly seizure severity questions and quality |
| | of life questions |
| Pain | Record events of elevated pain |
| | Store possible electrograpic records that correlate to pain |
| | sensation |
| | Record daily pain ratings |
| | Record medication logs |
| Depression | Record significant mood change event time/date |
| | Collect mood ratings |
| | Store possible electrographic records that correlate to |
| | depressed states |
| | Record daily depression rating |
| | Record quality of life questions |
| Movement | Record daily rating of movement disorder |
| Disorders | Store possible electrographic records that correlate to |
| | movement disorder symptoms. |
| | Record quality of life questions |
| Migraine | Record headache time/date |
| Headaches | Collect headache type and rating |
| | Store possible electrographic records that correlate to |
| | headache sensations |
| | Record quality of life questions |
| Cardiac | Record symptom time/date |
| monitoring | Store electrographic records that correlate to symptoms |
| | Record quality of life questions |

The applications listed in Table 1 are not intended to be exhaustive, or intended to limit embodiments of the invention in any way. These applications are listed as examples to establish utility. Table 2 lists some exemplary advantages associated with applications such as those listed in Table 1.

TABLE 2

| Activity | Advantage |
|---|---|
| Data | No longer need to keep paper event diaries unless additional |
| collection | event information is necessary or if the PER should |
| by patient | malfunction |
| | No longer need to use a large computer to interrogate their |
| | implanted device and synchronize the data with the centralized |
| | database |
| | No longer need to carry around a magnet for implantable |
| | medical device event marking |
| Data | No longer need to enter events into electronic forms |
| entry and | (verification of reported events may still be necessary) |
| analysis | Allows for real-time evaluation of clinical events along |
| by | with the electrographic correlates |
| clinician | Allows for daily tracking of patient's clinical progress |
| | On-demand reports may be generated listing all of the events |
| | logged by the patient for ease of review during clinic visits |
| Data | Only electrographic correlates of patient-reported clinical |
| storage | events need be designated as write-protected until uploaded - |
| by IMD | reduces memory size requirements for IMD and improves |
| | retention of clinically significant data |

Various forms of control logic can be used to implement the various features and functions associated with the invention. Such control logic can be implemented using hardware, software, or a combination thereof. For example, one or more servers, computing systems, controllers, processors, processing systems, ASICs, PLAs, and other computing devices, logic devices, modalities or components can be included to implement the desired features and functionality. Additionally, memory or other data and information communication and storage capacity can be included to facilitate operation of the device and communication among components.

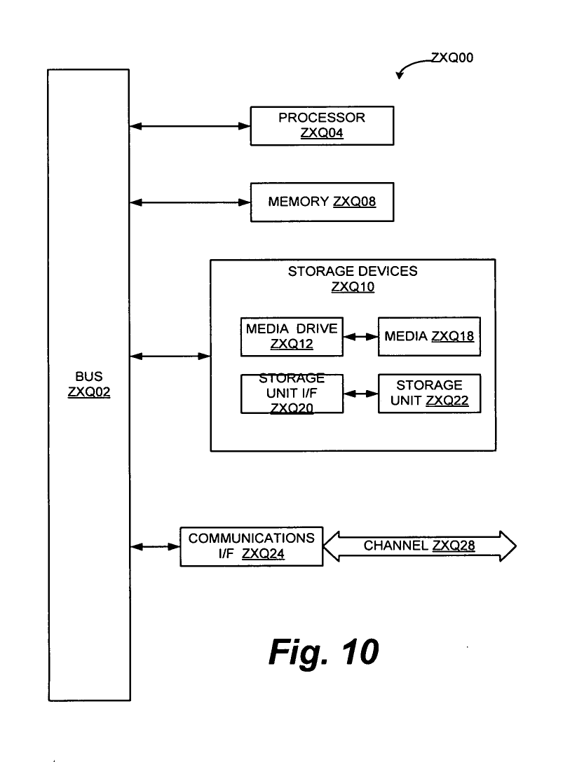

In fact, in one embodiment, these elements are implemented using a computing system capable of carrying out the functionality described with respect thereto. One such example computing system is shown in FIG. 10. Various embodiments are described in terms of this exemplary computing system ZXQ00. After reading this description, it will become apparent to a person skilled in the relevant art how to implement the invention using other computer systems or architectures.

Referring now to FIG. 10, computing system ZXQ00 may represent, for example, desktop, laptop and/or notebook computers; hand held computing devices (PDA's, cell phones, palmtops, etc.); mainframes, supercomputers, or servers; or any other type of special or general purpose computing device as may be desirable or appropriate for a given application or environment. Computing system ZXQ00 can include one or more processors, such as a processor ZXQ04. Processor ZXQ04 can be implemented using a general or special purpose processing engine such as, for example, a microprocessor, controller or other control logic. In the example illustrated in FIG. 10, processor ZXQ04 is connected to a bus ZXQ02 or other communication medium.

Computing system ZXQ00 can also include a main memory ZXQ08, preferably random access memory (RAM) or other dynamic memory, for storing information and instructions to be executed by processor ZXQ04. Main memory ZXQ08 may also be used for storing temporary variables or other intermediate information during execution of instructions to be executed by processor ZXQ04. Computing system ZXQ00 can likewise include a read only memory (“ROM”) or other static storage device(s) coupled to bus ZXQ02 for storing static information and instructions for processor ZXQ04.

The computing system ZXQ00 can also include information storage mechanism ZXQ10, which can include, for example, a media drive ZXQ12 and a removable storage interface ZXQ20. The media drive ZXQ12 can include a drive or other mechanism to support fixed or removable storage media. For example, a hard disk drive a floppy disk drive, a magnetic tape drive, an optical disk drive, a CD or DVD drive (R or RW), or other removable or fixed media drive. Storage media ZXQ18, can include, for example, a hard disk, a floppy disk, magnetic tape, optical disk, a CD or DVD, or other fixed or removable medium that is read by and written to by media drive ZXQ14. As these examples illustrate, the storage media ZXQ18 can include a computer usable storage medium having stored therein particular computer software or data.

In alternative embodiments, information storage mechanism ZXQ10 may include other similar instrumentalities for allowing computer programs or other instructions or data to be loaded into computing system ZXQ00. Such instrumentalities can include, for example, a removable storage unit ZXQ22 and an interface ZXQ20. Examples of such can include a program cartridge and cartridge interface, a removable memory (for example, a flash memory or other removable memory module) and memory slot, and other removable storage units ZXQ22 and interfaces ZXQ20 that allow software and data to be transferred from the removable storage unit ZXQ18 to computing system ZXQ00.

Computing system ZXQ00 can also include a communications interface ZXQ24. Communications interface ZXQ24 can be used to allow software and data to be transferred between computing system ZXQ00 and external devices. Examples of communications interface ZXQ24 can include a modem, a network interface (such as an Ethernet or other NIC card), a communications port (such as for example, a USB port), a PCMCIA slot and card, etc. Software and data transferred via communications interface ZXQ24 are in the form of signals which can be electronic, electromagnetic, optical or other signals capable of being received by communications interface ZXQ24. These signals are provided to communications interface ZXQ24 via a channel ZXQ28. This channel ZXQ28 can carry signals and can be implemented using a wireless medium, wire or cable, fiber optics, or other communications medium. Some examples of a channel can include a phone line, a cellular phone link, an RF link, a network interface, a local or wide area network, and other communications channels.

In this document, the terms “computer program medium” and “computer usable medium” are used to generally refer to media such as, for example, memory ZXQ08, storage device ZXQ18, a hard disk installed in hard disk drive ZXQ12, and signals on channel ZXQ28. These and other various forms of computer usable media may be involved in carrying one or more sequences of one or more instructions to processor ZXQ04 for execution. Such instructions, generally referred to as “computer program code” (which may be grouped in the form of computer programs or other groupings), when executed, enable the computing system ZXQ00 to perform features or functions of the present invention as discussed herein.

In an embodiment where the elements are implemented using software, the software may be stored in a computer program medium and loaded into computing system ZXQ00 using removable storage drive ZXQ14, hard drive ZXQ12 or communications interface ZXQ24. The control logic (in this example, software instructions or computer program code), when executed by the processor ZXQ04, causes the processor ZXQ04 to perform the functions of the invention as described herein.

While various embodiments of the present invention have been described above, it should be understood that they have been presented by way of example only, and not of limitation. Thus the breadth and scope of the present invention should not be limited by any of the above-described exemplary embodiments, but should be defined only in accordance with the following claims and their equivalents. Additionally, the invention is described above in terms of various exemplary embodiments and implementations. It should be understood that the various features and functionality described in one or more of the individual embodiments are not limited in their applicability to the particular embodiment with which they are described, but instead can be applied, alone or in some combination, to one or more of the other embodiments of the invention, whether or not such embodiments are described and whether or not such features are presented as being a part of a described embodiment.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives like “conventional,” “traditional,” “normal,” “standard,” “typical,” and terms of similar meaning should not be construed as limiting the item described to a given time period or to an item available as of a given time, but instead should be read to encompass conventional, traditional, normal, or standard technologies that may be available now or at any time in the future. Likewise, a group of items linked with the conjunction “and” should not be read as requiring that each and every one of those items be present in the grouping, but rather should be read as “and/or” unless expressly stated otherwise.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration. Indeed, alternative functional, logical or physical partitioning can be implemented to achieve the desired features and functionality of the present invention. Additionally, a multitude of different constituent module names other than those depicted herein can be applied to the various partitions. As an additional example, with regard to flow diagrams and their accompanying description, the order in which steps may be set forth shall not be interpreted as requiring that the operations take place in that particular order unless the context dictates otherwise.

1. A patient event recording device, comprising:
a first switch;
control logic operably coupled with the first switch;
a transmitter operably coupled with the control logic, the transmitter configurable to transmit to an implantable medical device responsive to an actuation of the first switch;
a receiver operably coupled with the control logic, the receiver configurable to receive transmissions from the implantable medical device, the received transmissions encoding medical device data;
a memory operably coupled with the control logic, the memory configurable to record at least a portion of the medical device data;
a first data communication interface operably coupled with the control logic, the first data communication interface configurable for coupling with a second communication data interface to relay the recorded data to a server; and
a power source operably configured to power the patient event recording device.

2. The device of claim 1, wherein the medical device data comprises a representation of an electrographic signal recorded by the implantable medical device.

3. The device of claim 1, wherein the medical device data further comprises diagnostic indicia for the implantable medical device.

4. The device of claim 1, further comprising a charger interface operably coupled to the power source, and wherein the power source comprises a rechargeable power cell.

5. The device of claim 1, further comprising:
a clock operably coupled with the control logic, the clock configurable to provide a time indication corresponding to the actuation of the first switch;
wherein the memory is further configurable to record the time indication; and
wherein the first data communication interface is further configurable to relay the time indication to the server.

6. The device of claim 5, further comprising:
at least one additional switch operably coupled to the control logic; and
a video display operably coupled to the control logic.

7. The device of claim 6, wherein the video display is configurable to display a time indication.

8. The device of claim 6, wherein the video display is configurable to display a prompt corresponding to a scheduled time indication.

9. The device of claim 8, wherein the displayed prompt comprises at least one of (i) a symptom question, (ii) a medication question, and (iii) an activity question.

10. The device of claim 9, wherein the control logic is further configurable to store the time indication and an indication of an actuation of at least one of the first switch or the at least one additional switch subsequent to the display of the prompt.

11. The device of claim 6, wherein the video display is configurable to display a prompt responsive to an actuation of at least one of the first switch or the at least one additional switch.

12. The device of claim 11, wherein the displayed prompt comprises at least one of (i) a symptom question, (ii) a medication question, and (iii) an activity question.

13. The device of claim 12, wherein the control logic is further configurable to store an indication of an actuation of at least one of the first switch or the at least one additional switch subsequent to the display of the prompt.

14. The device of claim 6, wherein the video display is configurable to display a prompt indicating an alarm condition consisting of at least one of (i) a scheduled server communication time indication, (ii) a scheduled medication administration time indication, (iii) a scheduled question response time indication, (iv) a memory capacity limit indication, and (v) a low power source indication.

15. The device of claim 5, further comprising an audio generator operably coupled to the control logic, and wherein the control logic is further configurable to actuate the audio generator indicating an alarm condition consisting of at least one of (i) a scheduled server communication time indication, (ii) a scheduled medication administration time indication, (iii) a scheduled question response time indication, (iv) a memory capacity limit indication, and (v) a low power source indication.

16. A method of operating a patient event recording device, comprising:
recording a time indication, corresponding to a first switch actuation, in a memory;
transmitting a first signal to an implantable medical device, the transmission being responsive to the first switch actuation;
receiving a second signal from the implantable medical device, the reception being responsive to the transmission of the first signal, the second signal encoding data comprising at least one of (i) a representation of an electrographic signal recorded by the implantable medical device, and (ii) diagnostic indicia for the implantable medical device; and
storing at least a portion of the encoded data in the memory.

17. The method of claim 16, further comprising:
displaying a prompt on a visual display responsive to the indication of the first switch actuation wherein the displayed prompt comprises at least one of (i) a symptom question, (ii) a medication question, and (iii) an activity question;
storing an indication of at least one switch actuation subsequent to the display of the prompt, in the memory.

18. The method of claim 16, further comprising transmitting the time indication and at least a portion of the at least a portion of the encoded data that are stored in the memory to a server.

19. The method of claim 18, further comprising receiving management data from the server and storing at least a portion of the management data in the memory.

20. The method of claim 19, further comprising displaying an indication of the stored management data on a video display.

21. The method of claim 19, further comprising actuating an audio generator responsive to the stored management data.

22. The method of claim 16, further comprising transmitting a signal to the implantable medical device indicating that a least a portion of data stored on the implantable medical device should be protected.

23. A system for recording and reporting patient events, comprising:
a patient event recorder comprising control logic, a memory, a clock, a transceiver configurable for bidirectional data communication with an implantable medical device, a first data communication interface, a power source, and at least one switch;
the implantable medical device, comprising a transceiver configurable for bidirectional data communication with the patient event recorder; and
a docking adapter, comprising a second data communication interface configurable for bidirectional data communication with the first data communication interface of the patient event recorder.

24. The system of claim 23 wherein the power source of the patient event recorder comprises a rechargeable power cell, the patient event recorder further comprises a charger interface operably coupled to the rechargeable power cell, and the docking adapter further comprises a charger configurable to charge the power cell via the charger interface.

25. The system of claim 23, wherein the second data communication interface is one of (i) a phone line modem, (ii) an Ethernet® adapter, (iii) a universal serial bus, (iv) a Bluetooth® modem; (v) an IEEE802® Standards-series modem, a (vi) TIA® Standards-based wireless data modem, and (vii) a 3GPP® Standards-based wireless data modem.

26. The system of claim 23, wherein the transceivers of both the implantable medical device and the patient event recorder are in compliance with one of (i) Bluetooth® Standards, and (ii) FCC regulations for 433 MHz ISM band operation.

27. The system of claim 23, further comprising a server configured for bidirectional communication with the second data communication interface.
